(19) United States
(12) Patent Application Publication
(10) Pub. No.: US 2016/0100974 A1
(43) Pub. Date: Apr. 14, 2016

(54) Nose Shaping Device

(71) Applicant: Young Han Joo, Jung-gu (KR)

(72) Inventor: Young Han Joo, Jung-gu (KR)

(21) Appl. No.: 14/513,614

(22) Filed: Oct. 14, 2014

(51) Int. Cl. A61F 5/08

(52) U.S. Cl. CPC A61F 5/08

(57) ABSTRACT

A nose shaping device is provided. The central portion touches and supports the ridge of the nose snuggly. Each of the two side arm portions extends sideways from the central portion and is configured for touching and applying a predetermined pressing force over the ridge side surface of the nose. Each of the two end portions is disposed at an end of a corresponding side arm portion and touches and supports the base of the ridge of the nose, and the central portion, the two side arm portion, and the two end portions are formed into an integral or monolithic piece. Each of the two connecting portions extends from a corresponding one of the two end portions. Each of the two ear-holding portions is disposed at an end of a corresponding one of the two connecting portions and configured to be held by an ear of a user.

### BACKGROUND OF THE INVENTION

The present invention relates to a nose shaping device. More particularly, this invention relates to a nose shaping device, which can help to adjust a shape of nose of user.

Accordingly, a need for a nose shaping device has been present for a long time considering the expansive demands in the everyday life these days. This invention is directed to solve these problems and satisfy the long-felt need.

### SUMMARY OF THE INVENTION

The present invention contrives to solve the disadvantages of the prior art.

An object of the invention is to provide a nose shaping device.

An aspect of the invention provides a nose shaping device comprising a central portion, two side arm portions, two end portions, two connecting portions, and two ear-holding portions.

The central portion is configured to touch and support the ridge of the nose snuggly.

Each of the two side arm portions extends sideways from the central portion and is configured for touching and applying a predetermined pressing force over the ridge side surface of the nose.

Each of the two end portions is disposed at an end of a corresponding side arm portion and configured for touching and supporting the base of the ridge of the nose, and the central portion, the two side arm portion, and the two end portions are formed into an integral or monolithic piece.

Each of the two connecting portions extends from a corresponding one of the two end portions.

Each of the two ear-holding portions is disposed at an end of a corresponding one of the two connecting portions and configured to be held by an ear of a user.

The integral or monolithic piece may be made of a material that can be bent with a force of predetermined magnitude, and maintain the bent shape with the external force removed and also under some other force of another predetermined external force, such as a pushing force from the side of side surface of the ridge of the nose.

The integral or monolithic piece may be made of aluminum, but not limited to it.

The integral or monolithic piece may be made of a flat material of given thickness, and the thickness is adjustable according to age, natural nose shape, and desired nose shape of the user.

The central portions has a partial shape of circle.

Each of the two side arm portions may have a shape of band, imposing a shaping force to the sides of the nose ridge.

Each of the two end portions may have a partial shape of circle.

Each of the two connecting portions may be made of elongated elastic material connecting one of the two end portions to a corresponding one of the two ear-holding portions.

A length of each of the two connecting portions may be adjustable.

Each of the two ear-holding portions may comprise a ring member.

The ring member may be made of a flexible string and a shape of the ring member is adjustable.

The nose shaping device may further comprise two latching portions, each of which being disposed between one of the two end portions and a corresponding one of the two connecting portions.

One end of each of the two latching portions may be fixed to a corresponding one of the two end portions and the other end thereof may be configured to be attached to a corresponding one of the connecting portions detachably.

The monolithic piece may have a triangular shape.

The advantages of the present invention are: (1) the nose shaping device is simple in structure and manufacturing; and (2) the nose shaping device according to the invention is convenient to use.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 is a top view showing a nose shaping device according to an embodiment of the invention;

FIG. 2 is a top view showing a nose shaping device according to another embodiment of the invention;

FIG. 3 is a top view showing a nose shaping device according to still another embodiment of the invention;

FIG. 4 is a top view showing a nose shaping device bent over a contour of nose according to still another embodiment of the invention;

FIG. 5 is a perspective view showing a nose shaping device according to still another embodiment of the invention;

FIG. 6 is a side view of FIG. 5; and

FIG. 7 is a front view showing a monolithic piece with two latching portions.

### DETAILED DESCRIPTION EMBODIMENTS OF THE INVENTION

Referring to the figures, the embodiments of the invention are described in detail.

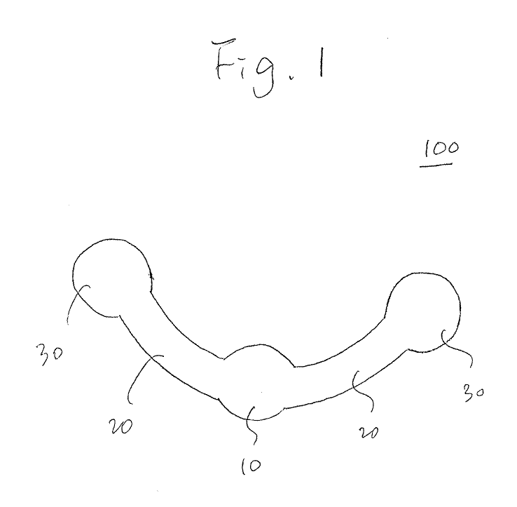

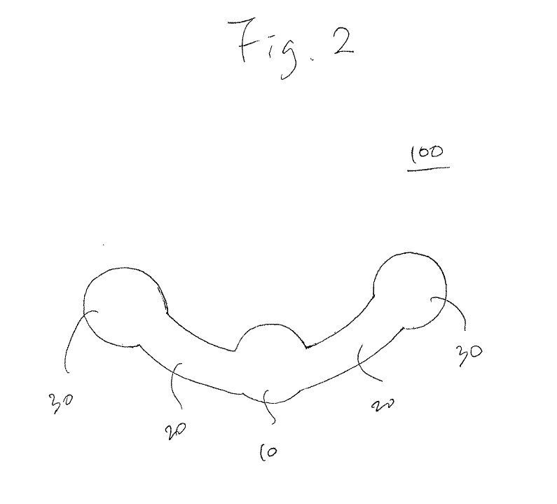

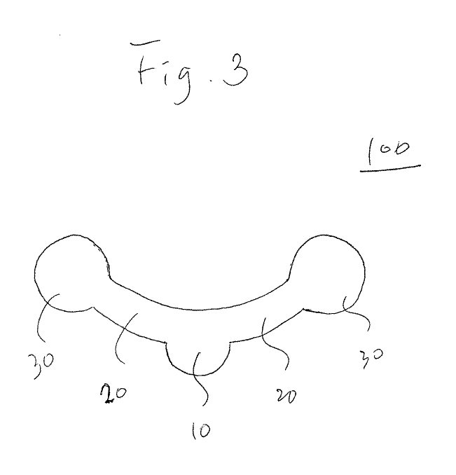

In FIGS. 1-4, each of the nose shaping device 100 includes a central portion 10, two side arm portions 20, and two end portions 30. An object of the invention is to provide a nose shaping device.

Sometimes, some people want their nose to be a little more pointed.

Without resorting to a painful or expensive measures such as cosmetic surgery, it can be done conveniently.

A nose can be pinched or restrained, imposing a desired shape. Basically, the nose is pinched, for example, by a pair of clothespins.

However, a conventional kinds of devices apply unconstrained persistent force to the nose, making it hurt.

Therefore, the invention provides a nose shaping device, which can relieve the pain accompanied by such conventional devices as clothespin and still help a lot.

As shown in FIGS. 1-3, each of nose shaping devices according to embodiments of the invention comprises:

a central portion configured to touch and support the ridge of the nose snuggly;

two side arm portions, each of which extending sideways from the central portion and being configured for touching and applying a predetermined pressing force over the ridge side surface of the nose; and

two end portions, each of which being disposed at an end of a corresponding side arm portion and configured for touching and supporting the base of the ridge of the nose,

wherein the central portion, the two side arm portion, and the two end portions are formed integrally or monolithically.

Also, the nose shaping device is made of a material that can be bent with a force of predetermined magnitude, and maintain the bent shape with the external force removed and also under some other force of another predetermined external force, such as a pushing force from the side of side surface of the ridge of the nose.

The material may include aluminum.

The nose shaping device can be made a flat material of given thickness. And, the thickness can be adjusted according to the age, natural nose shape, and desired nose shape.

The central portions may have a partial shape of circle. Such larger contacting area gives the user more convenient and safe contact at the ridge point of the nose.

Each of the two side arm portions may have a shape of band, imposing a shaping force to the sides of the nose ridge.

Each of the two base portions may have a partial shape of circle. Such larger contacting area gives the user more convenient and safe contact.

Of course, the shapes of the parts, especially the central portion and the two end portions, are not limited to circles. If they can provide snug contact and support to the nose of users, they can be of shapes of partially cut circle, polygon, ellipse, and rectangle.

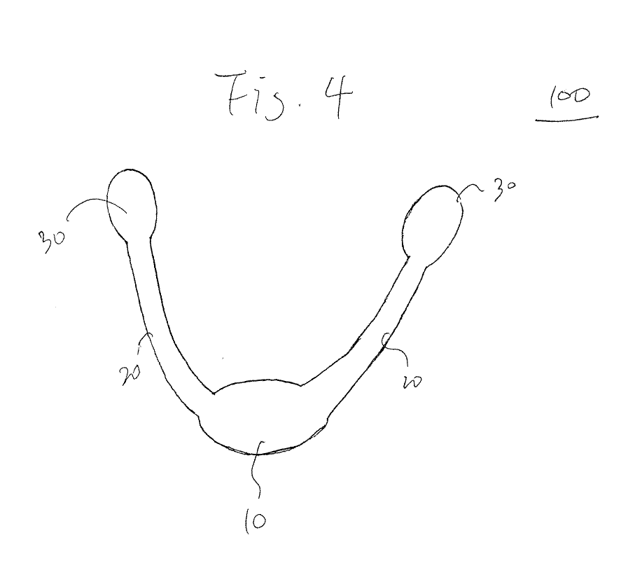

As shown in FIG. 4, the nose shaping device can be placed over the nose and pushed down against the ridge so as to bend the two end portions and the two side arm portions, following the sides of the nose ridge, into a desired cross-sectional shape of nose.

The thickness or resistant force of the material, for example aluminum, can be adjusted according to the pushing-out force from below the nose.

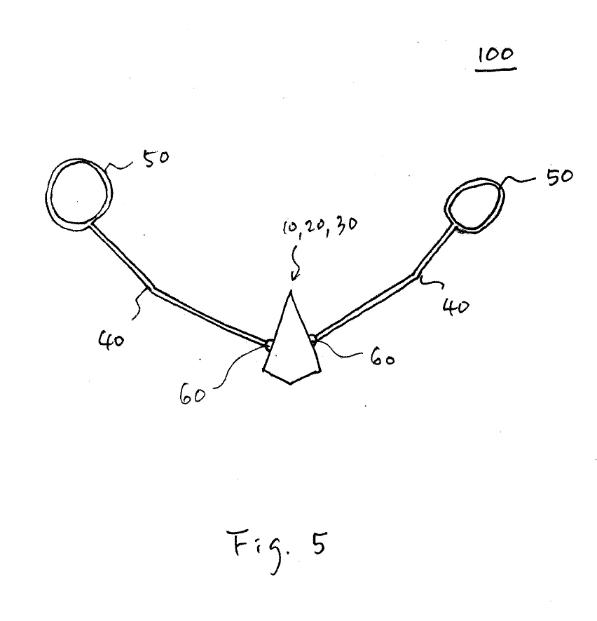

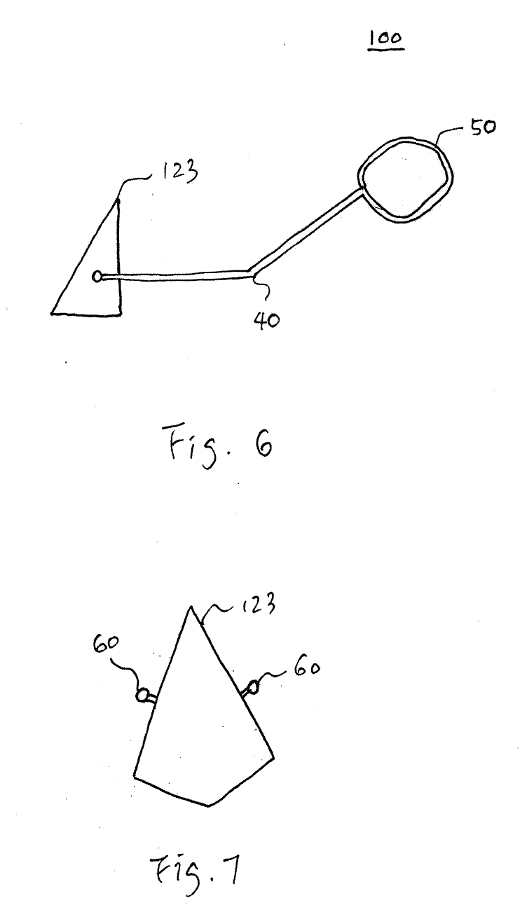

Another aspect of the invention as shown in FIGS. 5-7 provides a nose shaping device 100 comprising a central portion 10, two side arm portions 20, two end portions 30, two connecting portions 40, and two ear-holding portions 50.

The central portion 10 is configured to touch and support the ridge of the nose snuggly.

Each of the two side arm portions 20 extends sideways from the central portion 10 and is configured for touching and applying a predetermined pressing force over the ridge side surface of the nose.

Each of the two end portions 30 is disposed at an end of a corresponding side arm portion 20 and configured for touching and supporting the base of the ridge of the nose, and the central portion 10, the two side arm portion 20, and the two end portions 30 are formed into an integral or monolithic piece 123.

Each of the two connecting portions 40 extends from a corresponding one of the two end portions 30.

Each of the two ear-holding portions 50 is disposed at an end of a corresponding one of the two connecting portions 40 and configured to be held by an ear of a user.

The integral or monolithic piece 123 may be made of a material that can be bent with a force of predetermined magnitude, and maintain the bent shape with the external force removed and also under some other force of another predetermined external force, such as a pushing force from the side of side surface of the ridge of the nose.

The integral or monolithic piece 123 may be made of aluminum, but not limited to it.

The integral or monolithic piece 123 may be made of a flat material of given thickness, and the thickness is adjustable according to age, natural nose shape, and desired nose shape of the user.

The central portions 10 has a partial shape of circle.

Each of the two side arm portions 20 may have a shape of band, imposing a shaping force to the sides of the nose ridge.

Each of the two end portions 30 may have a partial shape of circle.

Each of the two connecting portions 40 may be made of elongated elastic material connecting one of the two end portions to a corresponding one of the two ear-holding portions 50.

A length of each of the two connecting portions 40 may be adjustable.

Each of the two ear-holding portions 50 may comprise a ring member.

The ring member may be made of a flexible string and a shape of the ring member is adjustable.

The nose shaping device 100 may further comprise two latching portions 60, each of which being disposed between one of the two end portions 30 and a corresponding one of the two connecting portions 40.

One end of each of the two latching portions 60 may be fixed to a corresponding one of the two end portions 30 and the other end thereof may be configured to be attached to a corresponding one of the connecting portions 40 detachably.

The monolithic piece 123 may have a triangular shape.

While the invention has been shown and described with reference to different embodiments thereof, it will be appreciated by those skilled in the art that variations in form, detail, compositions and operation may be made without departing from the spirit and scope of the invention as defined by the accompanying claims.

What is claimed is:

1. A nose shaping device comprising:
a central portion configured to touch and support the ridge of the nose snuggly;
two side arm portions, each of which extending sideways from the central portion and being configured for touching and applying a predetermined pressing force over the ridge side surface of the nose;
two end portions, each of which being disposed at an end of a corresponding side arm portion and configured for touching and supporting the base of the ridge of the nose, wherein the central portion, the two side arm portion, and the two end portions are formed into an integral or monolithic piece;
two connecting portions, each of which extending from a corresponding one of the two end portions; and
two ear-holding portions, each of which being disposed at an end of a corresponding one of the two connecting portions and configured to be held by an ear of a user.

2. The nose shaping device of claim 1, wherein the integral or monolithic piece is made of a material that can be bent with a force of predetermined magnitude, and maintain the bent shape with the external force removed and also under some other force of another predetermined external force, such as a pushing force from the side of side surface of the ridge of the nose.

3. The nose shaping device of claim 1, wherein the integral or monolithic piece is made of aluminum.

4. The nose shaping device of claim 1, wherein the integral or monolithic piece is made of a flat material of given thickness, and the thickness is adjustable according to age, natural nose shape, and desired nose shape of the user.

5. The nose shaping device of claim 1, wherein the central portions has a partial shape of circle.

6. The nose shaping device of claim 1, wherein each of the two side arm portions has a shape of band, imposing a shaping force to the sides of the nose ridge.

7. The nose shaping device of claim 1, wherein each of the two end portions has a partial shape of circle.

8. The nose shaping device of claim 1, wherein each of the two connecting portions is made of elongated elastic material connecting one of the two end portions to a corresponding one of the two ear-holding portions.

9. The nose shaping device of claim 8, wherein a length of each of the two connecting portions is adjustable.

10. The nose shaping device of claim 1, wherein each of the two ear-holding portions comprises a ring member.

11. The nose shaping device of claim 10, wherein the ring member is made of a flexible string and a shape of the ring member is adjustable.

12. The nose shaping device of claim 1, further comprising two latching portions, each of which being disposed between one of the two end portions and a corresponding one of the two connecting portions.

13. The nose shaping device of claim 12, wherein one end of each of the two latching portions is fixed to a corresponding one of the two end portions and the other end thereof is configured to be attached to a corresponding one of the connecting portions detachably.

14. The nose shaping device of claim 1, wherein the monolithic piece has a triangular shape.
